(19) United States
(12) Patent Application Publication
(10) Pub. No.: US 2024/0122461 A1
(43) Pub. Date: Apr. 18, 2024

(54) MULTI-BAND IMAGING FOR ENDOSCOPES

(71) Applicant: OmniVision Technologies, Inc., Santa Clara, CA (US)

(72) Inventor: Paul Wickboldt, Walnut Creek, CA (US)

(21) Appl. No.: 17/965,358

(22) Filed: Oct. 13, 2022

(51) Int. Cl. A61B 1/06; A61B 1/00; A61B 1/045

(52) U.S. Cl. CPC A61B 1/0638; A61B 1/000095; A61B 1/045; A61B 1/0655; A61B 1/0684; A61B 1/0002; A61B 1/00186

(57) ABSTRACT

A multi-band imager includes a first light source that illuminates an area of interest during a first time period and a second light source that illuminates the area of interest during a second time period, the second light source comprising a multi-band pass filter, an image sensor capturing first and second images during the first and second time periods. An enhanced image of the area of interest is generated by combining the first image and the second image.

### BACKGROUND

In their role as diagnostic tools, many medical endoscope applications benefit from an imaging system with the capability to discriminate between different types of tissue (normal, cancerous, polyps, lesions, etc.). This discrimination may be done using the spectral response of the light that is reflected from the tissue. Four general approaches have emerged: 1) Narrow-Band Illumination (NBI), which provides a mode where the light source can switch between white light and one or several narrow band LEDs; 2) Hyper-Spectral Imaging (HSI), which includes light sources that scan a wide spectrum with high resolution (e.g. every 10 nm); 3) Multi-spectral filter arrays (MSFAs) are imaging devices which use narrow band wavelengths for the color pixel filters to improve contrast of important image features; and 4) Identifying the type of tissue by fitting the full spectrum of reflected light to a model that provides the tissue's physical properties, for example, a neural network model.

NBI is useful when the user is seeking a clear image of underlying blood vessels and capillaries, which may allow for better identification of a range of conditions. NBI provides light from several LEDs with narrow-bands including at the absorption peaks of hemoglobin (˜415 nm and ˜565 nm). During use, the user may push a button that momentarily toggles the light source between white light and the narrow bands, so the blood vessels can be located with comparison to a normal image.

HSI captures a set of images, each from a narrow range of a total imaging spectrum. It provides maximal capability for the user to determine and use a combination of wavelengths to enhance specific features, but also generates a very large amount of data. Most work in HSI now focusses on data-driven studies and machine learning to determine the best spectral matches to identify tissue types. HSI is an expensive tool, requiring cart-sized electronics. Further, it requires optical fiber-fed light sources, limiting both the mechanical flexibility of the endoscope and the reduction of endoscope size. Another disadvantage of HSI is that each narrow spectral band captures very few photons. This makes taking video-rate images very challenging, as the image sensor may need very high integration times to get good photon count to achieve acceptable SNR.

MSFAs face the challenge that using narrow-band filters over the pixels creates a trade-off between image resolution and spectral resolution. This is a significant issue as the product trend has been to decrease sensor size to the limits of resolution. Further, users demand that the sensor always provide a “true” or “normal” image mode that is as close as possible to human perception. These conflicting demands of image resolution, spectral resolution, a normal image mode, and small sensor size make implementation of MSFAs challenging.

Identifying tissue types by fitting spectra to models of the tissue physics may someday prove reliable, but requires a tool that can capture the required spectral and spatial information at useful speeds and signal levels, that is compact and affordable and does not reduce endoscope performance.

### SUMMARY OF THE EMBODIMENTS

A multi-band imaging device provides higher spectral resolution of captured images to enable better identification of tissue features while still providing a product having a small size, required resolution, low cost and the ability to always provide true human-perception images.

In a first aspect, a multi-band imager includes a first light source that illuminates an area of interest during a first time period, a second light source comprising multi-band pass filter that illuminates the area of interest during a second time period and an image sensor receiving light from the first and second light sources reflected by the area of interest and capturing an image, and a memory for storing the first image while the area of interest is illuminated by the first light source. An enhanced image of the area of interest is generated by combining the first image and the second image.

In a second aspect, a multi-band imaging system the multi band imager, a processor; and a system memory storing machine readable instructions that, when executed by the processor, cause the processor to generate enhanced images of the area of interest by controlling the first light source to illuminate the area of interest during the first time period; controlling the image sensor to capture a first image while the area of interest is illuminated by the first light source; controlling the second light source to illuminate the area of interest during the second time period; controlling the image sensor to capture a second image while the area of interest is illuminated by the second light source; and generating an enhanced image of the area of interest by combining the first image and the second image.

In a third aspect, a method of generating an enhanced image of an area of interest includes controlling a first light source to illuminate the area of interest during a first time period and store it in a memory; controlling an image sensor to capture a first image while the area of interest is illuminated by the first light source; controlling a second light source to illuminate the area of interest during a second time period, the second light source including a multi-band pass filter; controlling the image sensor to capture a second image while the area of interest is illuminated by the second light source; and generating the enhanced image of the area of interest by combining the first image stored in the memory and the second image.

### BRIEF DESCRIPTION OF THE FIGURES

FIG. 1 is a graph showing a reflectance spectra comparison of five types of colon tissue across five wavelength bands, in an embodiment.

FIG. 2 is the graph of FIG. 1 showing a reflectance spectra comparison of five types of colon tissue across eight wavelength bands, in an embodiment.

FIG. 3 is a schematic diagram of a multi-band imager, in an embodiment.

FIGS. 4A and 4B show two different configurations for individual color filters for use in the multi-band imager of FIG. 3, in embodiments.

FIG. 5A is a graph of the characteristic response of an image sensor to full-spectrum visible white light, in embodiments.

FIG. 5B is a graph of the transmission spectra an IR cut off filter for use with an image sensor.

FIG. 5C is a graph of FIG. 5A with the IR cut off filter of FIG. 5B, in embodiments.

FIG. 5D is a graph of the transmission spectra of a multi-band pass filter used in the multi-band imager of FIG. 3, in embodiments.

FIGS. 6A and 6B are graphs showing the response of an image sensor to a single-band and multi-band pass light sources, respectively, in embodiments.

FIG. 7 is a graph showing the responses in FIGS. 6A and 6B combined to provide a spectral response with five spectral bands, in embodiments.

FIG. 8 is a flowchart illustrating a method for capturing multi-band images with an enhanced discrimination between features based on spectral response, in embodiments.

FIG. 9 is a block diagram of a multi-band imaging system, in embodiments.

### DETAILED DESCRIPTION OF THE EMBODIMENTS

The reflectance spectra of human tissues of interest tend to include broad spectral features. Changes in tissue as shown by changes in their spectra occur across broad bands. It has also been observed that all the spectra for tissue tend to include generally the same features, differing only in that the features may have different levels of intensity. This is true even across the different types of tissue (gastric, colon, airway, etc.).

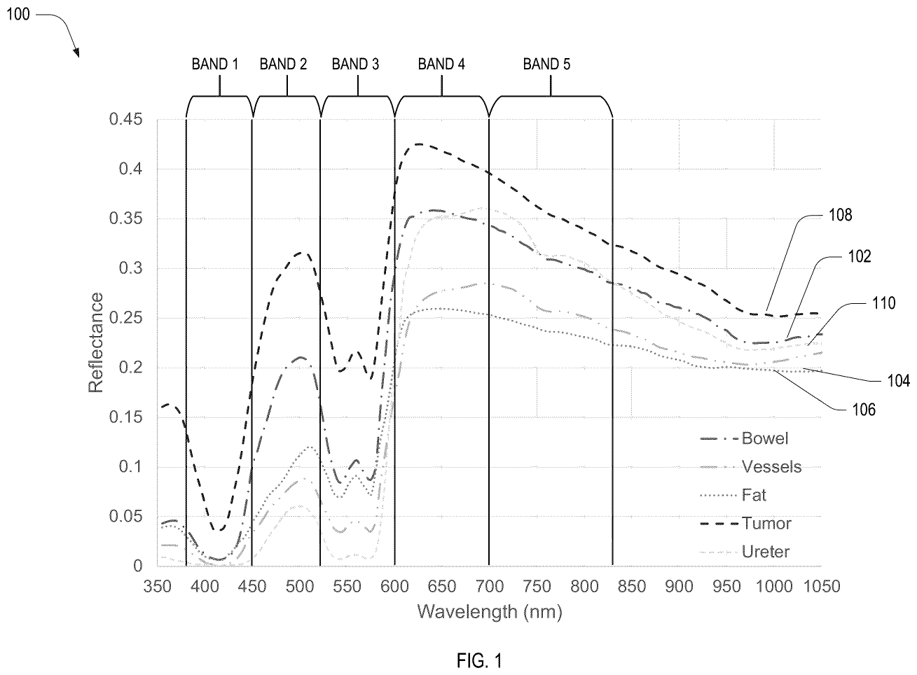

FIG. 1 is a graph 100 of a reflectance spectrum showing a spectral comparison of five types of colon tissue. For example, FIG. 1 shows spectra of bowel tissue at line 102, vessel tissue at line 104, fat tissue at line 106, tumor tissue at line 108, and ureter tissue at line 110. A comparison of lines 102, 104, 106, 108, and 110 shows that all the spectra include generally the same features at similar wavelengths, differing only in that the features may have different levels of intensity, or reflectance. Although colon tissue is shown in FIG. 1, similar spectra exist across different types of tissue such as gastric, airway, etc. All the spectra can be explained by a simple model that combines only a few features of the tissue: 1) the absorption of blood, 2) the collagen microstructures (fibrils), and 3) the thickness of the top (mucous) layer of the tissue. All the tissue spectra can be explained and simulated by adjusting the contributions of these three features and tuning their characteristics, including the oxygen content of the blood, the size of collagen scattering centers, and the relative content of each.

The features of the five spectra shown in FIG. 1 include a valley at approximately 410 nm, a peak at approximately 500 nm, a double valley at approximately 570 nm, a peak at approximately 610 nm, and a gradually sloping line at higher wavelengths. Each of the features spans at least 70 nm, therefore, a feature in tissue may be identified according to one of Band 1, Band 2, Band 3, Band 4, or Band 5. Although the spectra feature appearing in Band 3 near 570 nm may appear to be two features, these features are known to be due to a doublet, and may be identified together as a single feature.

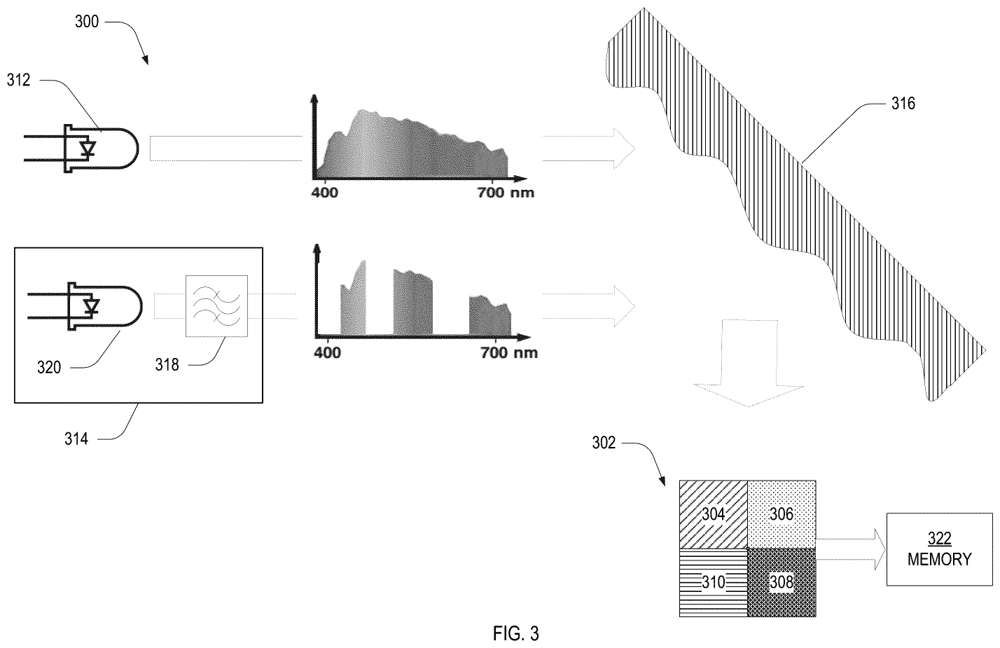

As illustrated in FIG. 1, detecting features of interest involves detecting a spectral response with bands as narrow as 70 nm, which is too small for standard low-cost color filter layers which have bands at least twice that width. To achieve the narrow band response, embodiments disclosed herein combine the use of pixel color filter layers, and a secondary light source that emits a multi-banded light. As discussed below in connection with FIG. 3, a multi-band imager includes one light source that is a standard white light LED. This source illuminates the tissue, which emits reflected light at its characteristic spectrum. The sensor module captures the image. With a standard white light source and color pixel CMOS, true color images and video can be captured.

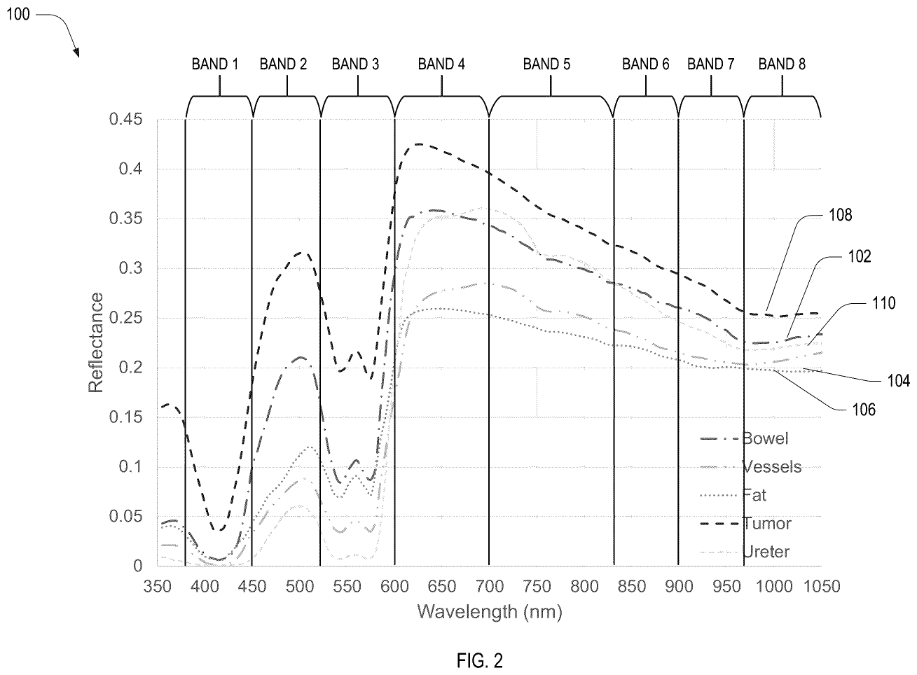

In embodiments, additional spectra such as near infrared (NIR), may be used to scan tissue. FIG. 2 shows graph 100 with three additional bands defined as Band 6, Band 7, and Band 8 across the NIR spectrum of approximately 750 nm-1050 nm. Embodiments discussed herein, may be understood to apply to spectra resulting from scanning an area of interest using any wavelength of electromagnetic radiation. The bands shown in FIGS. 1 and 2 are based, in part, on the different spectra determined from experimentation and the underlying physical models that explain the differences between spectra.

FIG. 3 is a schematic diagram of multi-band imager 300, which uses a combination of an image sensor 302 formed from an array of pixels having n pixel individual color filters (e.g., n=4 pixels) together with a 2-mode light source. Multi-band imager 300 is capable of generating images at up to 2× n (e.g., 8) spectral bands. In embodiments, image sensor 302 is an array of pixels grouped in 2×2 squares that capture image information at a point. Each 2×2 square is covered by four color filters: red filter 304, green filter 306, blue filter 308, and yellow filter 310 that correspond to respective pixels of the array of pixels. A 2×2 square of pixels captures color information in four channels. The color, or spectrum, at that point under white LED illumination that may be represented as (α, β, γ, δ). The wavelength value for α is captured by a pixel with red filter 304 at approximately 600-820 nm. The wavelength value for β is captured by a pixel with green filter 306 at approximately 450-680 nm. The wavelength value for γ is captured by a pixel with blue filter 308 at approximately 350-550 nm and the wavelength value for δ is captured by a pixel with a yellow filter 310 at approximately 450-820 nm.

Although the description above assumes that a is a value at a pixel with red filter 304, β is a value at a pixel with green filter 306, γ is a value at a pixel with blue filter 308, and δ is a value at a pixel with yellow filter 310, each pixel may be assigned a pixel value for all of the individual color filters based on a demosaicing process, as understood by one of ordinary skill in the art.

The 2-mode light source may be provided, for example, by first and second light sources 312 and 314. First light source 312 illuminates an area of interest in tissue 316 with a full white light spectrum, and second light source 314 comprising a light source 320 and a multi-band pass filter 318 having three or four wide bands also illuminates approximately the same area of tissue 316 as first light source 312. Light source 320 may be the same as first light source 312, in other words, one light source may provide both a full white light spectrum and a multi-band spectrum. Each of the bands passed by the multi-band pass filter 318 may be between approximately 50 nm and 150 nm wide, for example, and span wavelengths that partially overlap the wavelengths captured by individual color filters 304, 306, 308 and 310. In embodiments, multi-band pass filter 318 may be an optical filter such as a dichroic or interference filter.

In embodiments, light from first and second light sources 312 and 314 is reflected by tissue 316, recorded by image sensor 302, and combined to compute images at up to five unique spectral bands. Further computational processing may be used to enhance the contrast of features that have specific spectral responses. In embodiments, first light source 312 and light source 320 may be light-emitting diodes (LEDs).

For purposes of illustration, principles will be discussed herein in terms of the visible light spectrum and five bands as shown in FIG. 1 with pixel color filters similar to standard RGB and Yellow broad band filters that allow standard full color images. Image sensor 302 is depicted with four full pixels, however, individual color filters 304, 306, 308 and 310 form a 2×2 square that is part of a color filter array having a repeating pattern of individual color filters, each individual color filter being aligned to a respective pixel of the array of pixels.

To provide increased spectral resolution greater than what can be achieved with a single-band light source (“white light”) alone, a first image and a second image may be alternately captured using image sensor 302 using the first light source 312 with a full visible spectrum and second light source 314 with a multi-band spectrum, respectively. The captured first image is stored in a memory 322 prior to the capture of the second image. Optionally, the captured second image may be stored in memory 322 after the capture of the second image.

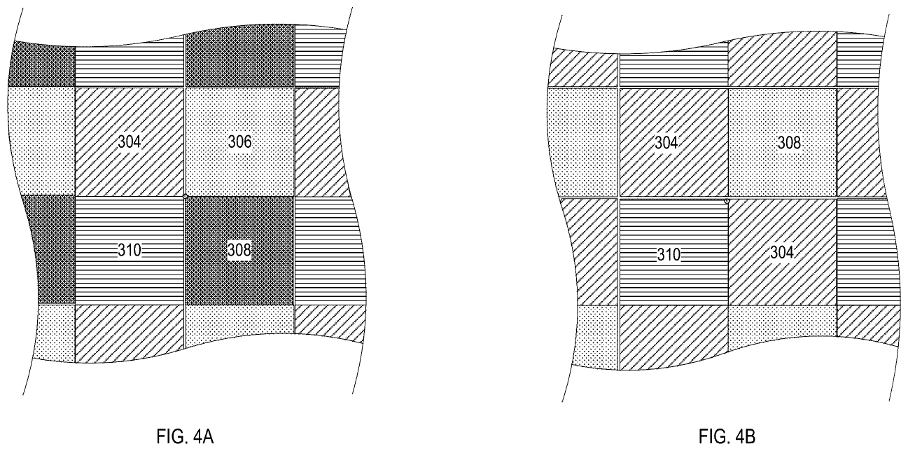

FIGS. 4A and 4B show two different configurations for individual color filters, in embodiments. FIG. 4A illustrates n=4 pixels with individual red filter 304, green filter 306, blue filter 308, and yellow filter 310. The individual color filters are arranged in a 2×2 square that are repeated across the array of pixels. FIG. 4B shows two red filters 304, a blue filter 308 and a yellow filter 310 arranged in a 2×2 square and repeated across an array of pixels. Other arrangements of individual color filters are contemplated.

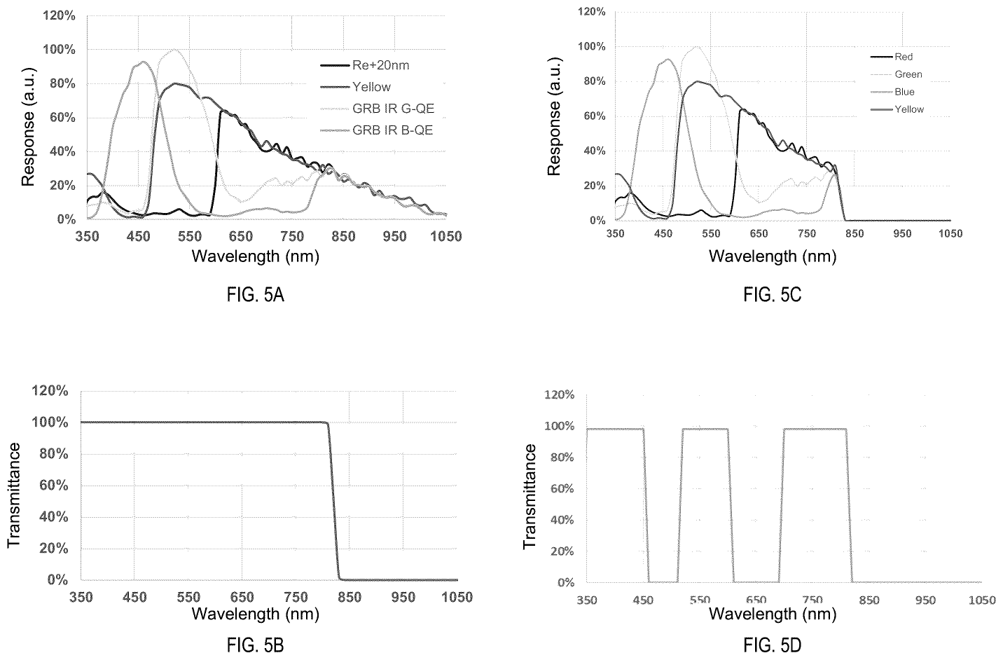

FIG. 5A is a graph of wavelength vs. absorbance that illustrates the characteristic response of image sensor 302 to full-spectrum visible white light from first light source 312. Four individual color filters are used, for RGB and Yellow. In embodiments, a range of wavelengths filtered by each individual color filter may be adjusted higher or lower by approximately 10-50 nm. For the five-band embodiment of FIG. 1, image sensor 302 also includes an IR cut filter with a transmission spectra as shown in FIG. 5B and a cut-off at 820 nm, resulting in the response shown in the graph of FIG. 5C. In other embodiments, different arrangements of color filters may be used. For example, the RGBY filter pattern shown in FIG. 4A may be replaced with a RYYB filter where filter 304 is red, filters 306 and 308 are yellow and filter 310 is blue. The RGBY filter patter may also be replaces with an RRYB pattern where filters 304 and 306 are red, filter 308 is yellow and filter 310 is blue. Other filter patterns are contemplated.

FIG. 5D shows transmission spectra of multi-band pass filter 318 of second light source 314. In embodiments, multi-band pass filter 318 includes three pass bands at wavelengths below the cut-off wavelength of the IR cut filter. Band edges are at 450 nm, 520 nm, 600 nm, 700 nm, and 820 nm, chosen to align well with the bands of interest in FIG. 1. In embodiments, band edges may differ. When an area of interest includes significant features having spectral responses at different wavelengths, both the pass band wavelengths, and the filtered wavelengths of the individual color filters may be adjusted.

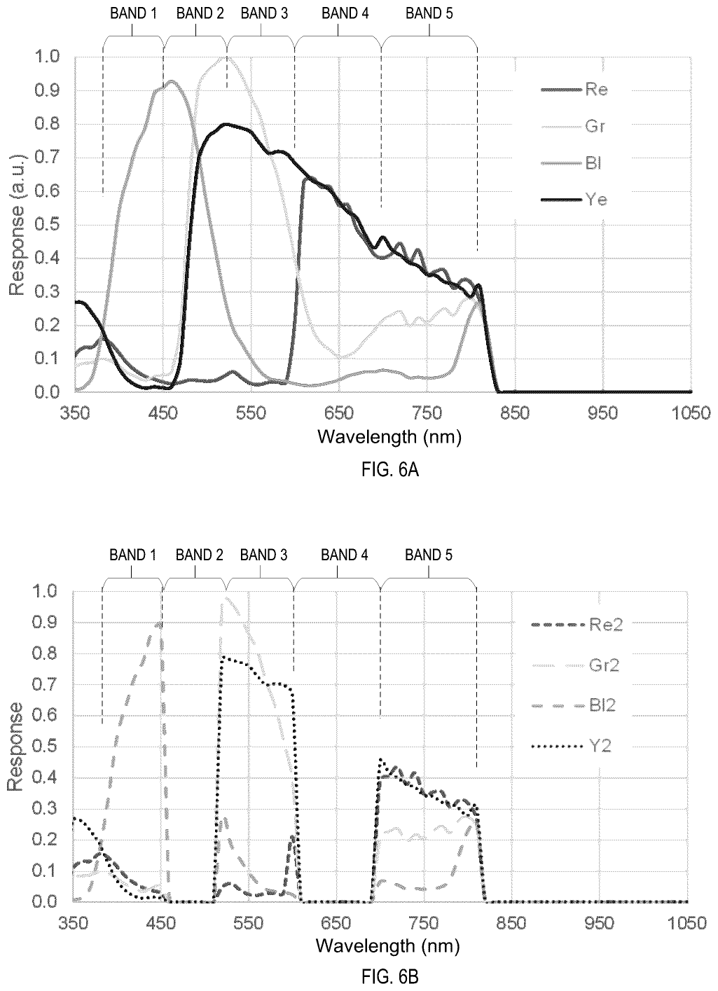

FIG. 6A is a graph showing the response of image sensor 302 to a single-band light source such as first light source 312. FIG. 6B is a graph showing the response of image sensor 302 to second light source 314. Bands 1-5 of FIG. 1 are shown to illustrate the correspondence between bands of multi-band pass filter 318 and features of an area of interest in an imaged tissue. In embodiments, the response from first light source 312 may be used to construct a true color image with responses close to the primary standard observer responses.

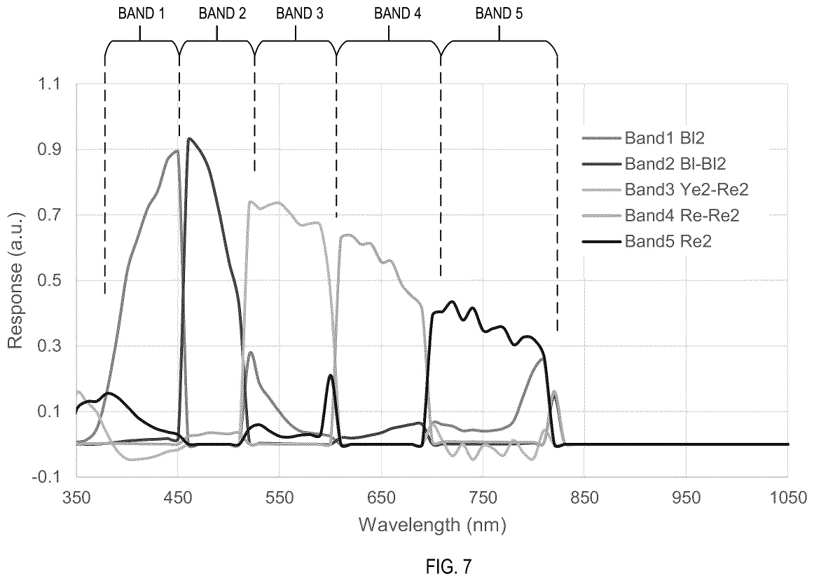

FIG. 7 illustrates how the responses in FIGS. 6A and 6B may be combined to provide a spectral response with five spectral bands. Calculations involve subtractions of only two pixel responses, which would limit the accumulation of noise effects. As noted above, when image sensor 302 is illuminated by first light source 312, image sensor 302 captures color information (characteristic spectrum) in 4 channels (4 bands), designated as (α, β, γ, δ). Values for (α, β, γ, δ) across all 2×2 squares of pixels of image sensor 302 represents a first image. The captured first image is stored in memory 322. Then, when image sensor 302 is illuminated with multi-band light from second light source 314, the value detected from RGBY pixels of image sensor 302 is designated as (α′, β′, γ′, δ′). Values for (α′, β′, γ′, δ′) across all 2×2 squares of pixels of image sensor 302 represents a second image. Optionally, the captured second image is stored in memory 322.

Generating an enhanced image with spectral response greater than that achievable with white light alone includes subtractions of values for (α, β, γ, δ) and (α′, β′, γ′, δ′) to get color information (characteristic spectrum) in 5 channels (5 bands), designated as (p, q, r, s, t), according to the following equations:

p=γ′ (1)

q=γ−γ′ (2)

r=δ′−α′ (3)

s=α−α′ (4)

t=α′ (5)

Subtractions occur for each set of 2×2 pixels. Band 1 of FIG. 7 is given by the equation for p, in approximately 350-450 nm, Band 2 is given by the equation for q, in approximately 450-500 nm, Band 3 is given by the equation for r, in approximately 500-600 nm, Band 4 is given by the equation for s, in approximately 600-700 nm, and Band 5 is given by the equation for t, in approximately 700-820 nm.

FIG. 7 also shows how the spectral bands captured by image sensor 302 correspond to Bands 1-5 from FIG. 1 that were selected for their usefulness in looking at the changing features in the human tissue spectra. In a full endoscopic system, this spectral data may be compiled different ways to form images where features that correspond to different combinations of spectral bands are enhanced to identify different tissue types or conditions.

In embodiments, multi-band imager 300 may be used in different modes to suit the users' needs. A true-image only mode would utilize images captured when first light source 312 illuminates tissue 316. Modes that utilize the spectral image may be taken in a spectrally enhanced mode where the white-light images captured with first light source 312 and banded light images captured with second light source 314 are captured alternately then processed. Multi-band imager 300 may capture and process both still images and video.

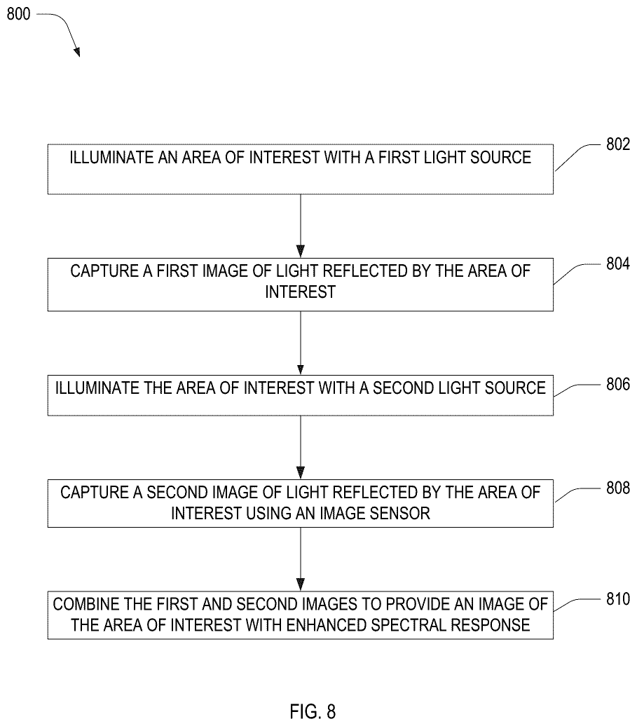

FIG. 8 is a flowchart illustrating a method 800 for capturing multi-band images with an enhanced discrimination between features based on spectral response.

Step 802 includes illuminating an area of interest with a first light source. In an example of step 802, an area of interest in tissue 316 is illuminated with first light source 312 during a first time period. Light from first light source 312 is reflected from tissue 316 toward image sensor 302.

Step 804 includes capturing a first image of light reflected by the area of interest. In an example of step 804, reflected light from tissue 316 is captured by image sensor 302 during or immediately after the first time period. The captured first image is stored in memory 322.

Step 806 includes illuminating an area of interest with a second light source. In an example of step 806, an area of interest in tissue 316 is illuminated with second light source 314 during a second time period. Light from first light source 312 is reflected from tissue 316 toward image sensor 302.

Step 808 includes capturing a second image of light reflected by the area of interest. In an example of step 808, reflected light from tissue 316 is captured by image sensor 302 during or immediately after the second time period. The captured second image is optionally stored in memory 322.

Step 810 includes combining the first and second images to provide an image of the area of interest having an enhanced spectral response.

To obtain spectral resolution higher than that obtainable with a single-band light source, images may be alternately captured using both first light source 312 and second light source 314 having multi-band pass filter 318. In embodiments, both single images and videos are captured. When capturing video, a 60 fps (frame per second) capture rate may be used, while alternately illuminating each frame at 30 fps with the two light sources. The responses of these two types of images are then combined to achieve spectral images.

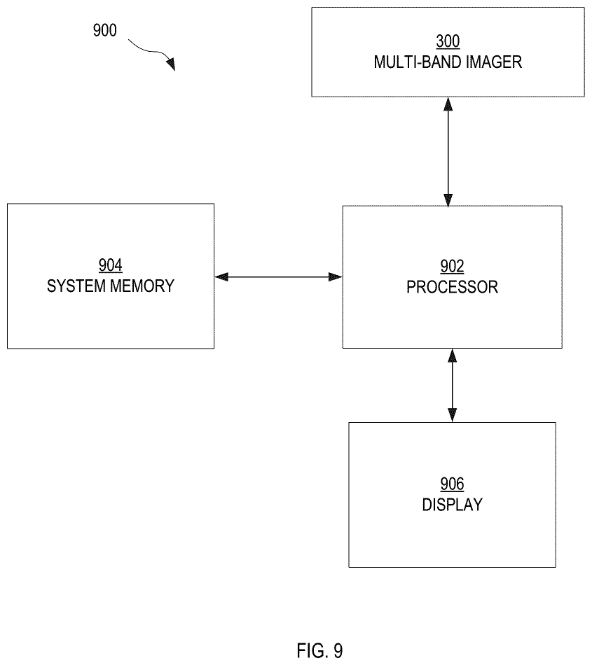

FIG. 9 is a block diagram of a multi-band imaging system 900, which includes multi-band imager 300, processor 902, system memory 904, and display 906. Multi-band imager 300 is controlled by processor 902 executing machine-readable instructions stored in system memory 904 to cause the processor to perform the method of FIG. 8. Display 906 displays a graphical user interface for operating multi-band imaging system 900, images or video captured by multi-band imager 300, and any other information necessary for operating the system.

System memory 904 may be transitory and/or non-transitory and may include one or both of volatile memory (e.g., SRAM, DRAM, computational RAM, other volatile memory, or any combination thereof) and non-volatile memory (e.g., FLASH, ROM, magnetic media, optical media, other non-volatile memory, or any combination thereof). Part or all of system memory 904 may be integrated into processor 902. Memory 322 may be included in system memory 904.

A multi-band imager as disclosed herein has the advantages of NBI in that it maintains full image resolution, provides a good video frame rate, uses a local small LED light source, and provides a standard RGB image, but also has the spectral-matching capability of HSI without high costs. Multi-band imaging systems disclosed here take advantage of the fact that the variations of the spectrum between different types of human tissue take place over broad spectral regions (>70 nm) and may not require the fine spectral resolution of HSI.

While a specific embodiment has been discussed herein, other embodiments are possible. Specifically, more bands may be created by increasing the number of bands in the band-pass filter, and choosing corresponding color filters for the pixels. As noted above, with a four-pixel combination, up to eight bands are possible. This could be expanded into the NIR region of the spectrum as shown in FIG. 2 where tissue features may be identified using the slope and structure of the tissue spectra in this region.

### Combinations of Features

Features described above as well as those claimed below may be combined in various ways without departing from the scope hereof. The following enumerated examples illustrate some possible, non-limiting combinations:

(A1) A multi-band imager includes a first light source that illuminates an area of interest during a first time period, a second light source comprising multi-band pass filter that illuminates the area of interest during a second time period, an image sensor capturing a first image when receiving light from the first light source reflected by the area of interest and capturing a second image when receiving light second light source reflected by the area of interest; and a memory for storing the first image while the area of interest is illuminated by the first light source, wherein an enhanced image of the area of interest is generated by combining the first image and the second image.

(A2) In multi-band imager (A1), the image sensor further may comprise an array of pixels and a color filter array of individual color filters corresponding to respective pixels of the array of pixels.

(A3) In multi-band imager (A2), the individual color filters include red, green, yellow and blue filters arranged in a 2×2 square repeating across the array of pixels.

(A4) In multi-band imager (A2), the individual color filters include two red filters, a yellow filter and a blue filter arranged in a 2×2 square repeating across the array of pixels.

(A5) In multi-band imager (A2), the individual color filters include a red filter, two yellow filters and a blue filter arranged in a 2×2 square repeating across the array of pixels.

(A6) In multi-band imager (A1), the image sensor further comprises an array of pixels and a color filter array and bands in the multi-band pass filter are selected to correspond to the color filter array to increase a spectral resolution of the enhanced image.

(A7) In any of multi-band imagers (A1)-(A6), the first and second light sources generate light in a visible light spectrum.

(A8) In any of multi-band imagers (A1)-(A7), the first light source is a light-emitting diode (LED) and the second light source comprises an LED.

(A9) In any of multi-band imagers (A1)-(A8), the first and second light sources generate light in visible and near infrared (NIR) spectrums.

(A10) In any of multi-band imagers (A1)-(A9), the multi-band pass filter is an interference filter.

(B1) A multi-band imaging system includes any of multi-band imagers (A1)-(A10), a processor, and a system memory storing machine readable instructions that, when executed by the processor, cause the processor to generate enhanced images of the area of interest by controlling the first light source to illuminate the area of interest during the first time period, controlling the image sensor to capture a first image while the area of interest is illuminated by the first light source, controlling the second light source to illuminate the area of interest during the second time period, controlling the image sensor to capture a second image while the area of interest is illuminated by the second light source, and generating an enhanced image of the area of interest by combining the first image and the second image.

(B2) In system (B1), the first light source is a light-emitting diode (LED) and the second light source comprises an LED.

(B3) In either of systems (B1) or (B2), wherein the image sensor further comprises an array of pixels and a color filter array of individual color filters corresponding to respective pixels of the array of pixels.

(B4) In system (B3), the individual color filters include red, green, yellow and blue filters arranged in a 2×2 square repeating across the array of pixels.

(B5) In system (B3), the individual color filters include two red filters, a yellow filter and a blue filter arranged in a 2×2 square repeating across the array of pixels.

(B6) In system (B3), the individual color filters include a red filter, two yellow filters and a blue filter arranged in a 2×2 square repeating across the array of pixels.

(B7) In either of systems (B1) or (B2), the image sensor further comprises an array of pixels and a color filter array and the multi-band pass filter comprises a plurality of bands selected to correspond to the individual color filters to increase a spectral resolution of the enhanced image.

(B8) In any of systems (B1)-(B7), the first and second light sources generate light in a visible light spectrum.

(B9) In any of systems (B1)-(B7), the first and second light sources generate light in visible and near infrared (NIR) spectrums.

(C1) A method of generating an enhanced image of an area of interest, including controlling a first light source to illuminate the area of interest during a first time period, controlling an image sensor to capture a first image while the area of interest is illuminated by the first light source and store it in a memory, controlling a second light source to illuminate the area of interest during a second time period, the second light source including a multi-band pass filter, controlling the image sensor to capture a second image while the area of interest is illuminated by the second light source; and generating the enhanced image of the area of interest by combining the first image stored in the memory and the second image.

(C2) In the method of (C1), storing the second image in the memory.

(C3) In either of methods (C1) or (C2), the multi-band pass filter is an interference filter.

(C4) In any of methods (C1)-(C3), generating the enhanced image further comprises subtracting a pixel response of the image sensor when the area of interest is illuminated by the second light source from a pixel response of the image sensor when the area of interest is illuminated by the first light source.

Changes may be made in the above methods and systems without departing from the scope hereof. It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

1. A multi-band imager comprising:
a first light source that illuminates an area of interest during a first time period;
a second light source comprising multi-band pass filter that illuminates the area of interest during a second time period;
an image sensor capturing a first image when receiving light from the first light source reflected by the area of interest and capturing a second image when receiving light second light source reflected by the area of interest; and
a memory for storing the first image while the area of interest is illuminated by the first light source;
wherein an enhanced image of the area of interest is generated by combining the first image and the second image.

2. The multi-band imager of claim 1, wherein the image sensor further comprises an array of pixels and a color filter array of individual color filters corresponding to respective pixels of the array of pixels.

3. The multi-band imager of claim 2, wherein the individual color filters include red, green, yellow and blue filters arranged in a 2×2 square repeating across the array of pixels.

4. The multi-band imager of claim 2, wherein the individual color filters include two red filters, a yellow filter and a blue filter arranged in a 2×2 square repeating across the array of pixels.

5. The multi-band imager of claim 2, wherein the individual color filters include a red filter, two yellow filters and a blue filter arranged in a 2×2 square repeating across the array of pixels.

6. The multi-band imager of claim 1, wherein the image sensor further comprises an array of pixels and a color filter array and bands in the multi-band pass filter are selected to correspond to the color filter array to increase a spectral resolution of the enhanced image.

7. The multi-band imager of claim 1, wherein the first and second light sources generate light in a visible light spectrum.

8. The multi-band imager of claim 1, wherein the first light source is a light-emitting diode (LED) and the second light source comprises an LED.

9. The multi-band imager of claim 1, wherein the first and second light sources generate light in visible and near infrared (NIR) spectrums.

10. The multi-band imager of claim 1, wherein the multi-band pass filter is an interference filter.

11. A multi-band imaging system comprising:
a multi-band imager comprising:
a first light source that illuminates an area of interest during a first time period;
a second light source comprising multi-band pass filter that illuminates the area of interest during a second time period;
an image sensor receiving light from the first and second light sources reflected by the area of interest and capturing an image; and
a memory; and
a processor; and
a system memory storing machine readable instructions that, when executed by the processor, cause the processor to generate enhanced images of the area of interest by:
controlling the first light source to illuminate the area of interest during the first time period;
controlling the image sensor to capture a first image while the area of interest is illuminated by the first light source;
controlling the second light source to illuminate the area of interest during the second time period;
controlling the image sensor to capture a second image while the area of interest is illuminated by the second light source; and
generating an enhanced image of the area of interest by combining the first image and the second image.

12. The system of claim 11, wherein the first light source is a light-emitting diode (LED) and the second light source comprises an LED.

13. The system of claim 11, wherein the image sensor further comprises an array of pixels and a color filter array of individual color filters corresponding to respective pixels of the array of pixels.

14. The system of claim 13, wherein the individual color filters include red, green, yellow and blue filters arranged in a 2×2 square repeating across the array of pixels.

15. The system of claim 13, wherein the individual color filters include two red filters, a yellow filter and a blue filter arranged in a 2×2 square repeating across the array of pixels.

16. The system of claim 13, wherein the individual color filters include a red filter, two yellow filters and a blue filter arranged in a 2×2 square repeating across the array of pixels.

17. The system of claim 11, wherein the image sensor further comprises an array of pixels and a color filter array and the multi-band pass filter comprises a plurality of bands selected to correspond to the individual color filters to increase a spectral resolution of the enhanced image.

18. The system of claim 11, wherein the first and second light sources generate light in a visible light spectrum.

19. The system of claim 11, wherein the first and second light sources generate light in visible and near infrared (NIR) spectrums.

20. A method of generating an enhanced image of an area of interest, comprising:
controlling a first light source to illuminate the area of interest during a first time period;
controlling an image sensor to capture a first image while the area of interest is illuminated by the first light source and store it in a memory
controlling a second light source to illuminate the area of interest during a second time period, the second light source including a multi-band pass filter;
controlling the image sensor to capture a second image while the area of interest is illuminated by the second light source; and
generating the enhanced image of the area of interest by combining the first image stored in the memory and the second image.

21. The method of claim 20, further comprising storing the second image in the memory.

22. The method of claim 20, wherein the multi-band pass filter is an interference filter.

23. The method of claim 20, wherein generating the enhanced image further comprises subtracting a pixel response of the image sensor when the area of interest is illuminated by the second light source from a pixel response of the image sensor when the area of interest is illuminated by the first light source.

24. A multi-band imager comprising:
a first light source that illuminates an area of interest during a first time period;
a second light source comprising multi-band pass filter emitting multi-banded light that illuminates the area of interest during a second time period;
an image sensor capturing a first image when receiving light from the first light source reflected by the area of interest and capturing a second image when receiving light second light source reflected by the area of interest; and
a memory for storing the first image while the area of interest is illuminated by the first light source;
wherein an enhanced image of the area of interest is generated by combining the first image and the second image.

25. The multi-band imager of claim 24, wherein the first light source and the second light source are LEDs.

26. A multi-band imaging system comprising:
a multi-band imager comprising:
a first light source that illuminates an area of interest during a first time period;
a second light source comprising multi-band pass filter emitting multi-banded light that illuminates the area of interest during a second time period;
an image sensor receiving light from the first and second light sources reflected by the area of interest and capturing an image; and
a memory; and
a processor; and
a system memory storing machine readable instructions that, when executed by the processor, cause the processor to generate enhanced images of the area of interest by:
controlling the first light source to illuminate the area of interest during the first time period;
controlling the image sensor to capture a first image while the area of interest is illuminated by the first light source;
controlling the second light source to illuminate the area of interest during the second time period;
controlling the image sensor to capture a second image while the area of interest is illuminated by the second light source; and
generating an enhanced image of the area of interest by combining the first image and the second image.

27. The multi-band imaging system of claim 26, wherein the first light source and the second light source are LEDs.

28. A method of generating an enhanced image of an area of interest, comprising:
controlling a first light source to illuminate the area of interest during a first time period;
controlling an image sensor to capture a first image while the area of interest is illuminated by the first light source and store it in a memory;
controlling a second light source to illuminate the area of interest during a second time period, the second light source including a multi-band pass filter emitting multi-banded light;
controlling the image sensor to capture a second image while the area of interest is illuminated by the second light source; and
generating the enhanced image of the area of interest by combining the first image stored in the memory and the second image.

29. The method of claim 28, wherein the first light source and the second light source are LEDs.
